(19) United States
(12) Patent Application Publication
(10) Pub. No.: US 2006/0204442 A1
(43) Pub. Date: Sep. 14, 2006

(54) Magnetically targetable particles comprising magnetic components and biocompatible polymers for site-specific delivery of biologically active agents

(75) Inventors: Gilles H. Tapolsky, Boulder, CO (US); Yuhua Li, Superior, CO (US); Yuyuan Jiao, Aurora, CO (US)

Correspondence Address: JENNIFER M MCCALLUM, PH D, ESQ;THE MCCALLUM LAW FIRM, LLC, 685 BRIGGS STREET, PO BOX 929, ERIE, CO, 80516, US

(73) Assignee: Bankruptcy Estate of Ferx, Inc., Arvada, CO (US)

(21) Appl. No.: 10/571,210

(22) PCT Filed: Sep. 11, 2004
(86) PCT No.: PCT/US04/29670

§ 371 Date: Mar. 8, 2006

Related U.S. Application Data
(60) Provisional application No. 60/502,737, filed on Sep. 12, 2003.

(51) Int. Cl. A61K 49/10; A61K 9/14

(52) U.S. Cl. 424/9.32; 424/489

(57) ABSTRACT

The invention relates to magnetically targetable particles comprising at least one magnetic component The particles are capable of delivering selectively to a site or organ a biologically active substance for in vivo medical diagnosis and/or treatment. The particles are prepared by many processes such as encapsulation processes. Also described are methods for making the particles, methods for localized in vivo delivery of a biologically active agent utilizing the particles, a kit for the administration of the particles, as well as a method for sterilizing the particles.

### FIELD OF THE INVENTION

This invention relates to compositions, methods of manufacture and methods of use for magnetically targetable particles that are capable of carrying biologically active compounds. These particles can be targeted to a specific site in the body as a therapeutic treatment for diseases, as a diagnostic aid, or as a bifunctional composition capable of acting as both a diagnostic and therapeutic agent.

### BACKGROUND OF THE INVENTION

The ability to selectively target therapeutic agents to a desired site within a mammalian body is an ongoing challenge. The targeted delivery of biologically active agents would enable enhancement of therapeutic activity of drugs while minimizing systemic side effects. Magnetic carrier compositions for treating various diseases have been previously described, and include compositions which are targeted to a specific location. Unfortunately, these compositions have demonstrated little therapeutic benefit. (Widder and Senyei, U.S. Pat. Nos. 4,247,406 and 4,357,259; Lieberman et al., U.S. Pat. No. 4,849,209; Schroder et al., U.S. Pat. No. 4,501,726; Chang, U.S. Pat. No. 4,652,257; Mirell, U.S. Pat. No. 4,690,130, and Kirpotin et al., U.S. Pat. No. 5,411,730).

Magnetic micro- or nano-spheres are of great interest in various fields of biotechnology and medicine. Currently, magnetic particles are used in vitro for separation of biochemical products (Margel et al., J. Cell Sci. 56:157-175 (1982); and Hedrum et al., PCR Methods Applications 2:167-171 (1992)) and cells (Kemshead et al., Br. J. Cancer, 54:771-778 (1986); and DeRosa et al., Haematologica 76:37-40, 75-84 (1992)) as well as for DNA detection. (Debuire et al., Clin. Cheni. 39:1682-1685 (1993); Suzuli et al., J. Virol. Meth. 41:341-350 (1993)). Magnetic responsive particles have been shown to increase the contrast in magnetic resonance imaging (MRI) when applied in the form of nanoparticles (Pouliquen et al., Magn. Resoji. Med. 24:75-84 (1992)), starch microspheres (Fahlvik et al., Invest. RadioL 25:793-797 (1990)), or magnetic particles. (Van Beers et al., Eur. J. Rad. 14:252 257 (1992); Oksendal et al., Acta Radiologica 34:187-193 (1993)). All of the nano- and micro-particles described in the above references are made using iron oxide (magnetite) as the magnetic responsive material and have specific requirements regarding particle size and magnetic susceptibility for separation and imaging applications. Although these magnetite-based particles have shown some success in separation and imaging applications, such compositions have not been demonstrated to be practical and/or effective for therapeutic use. Indeed, these particles have a magnetic susceptibility that does not allow for effective magnetic targeting and they lack the adequate capacity for delivering therapeutically relevant amounts of a biologically active agent. (Widder et al., Proc. Soc Exp. Biol. Med. 58:141 (1978); Widder et al., Eur. J. Cancer Clin. Oncol. 19:135 (1983); Pulfer et al., “Targeting Magnetic Microspheres to Brain tumors”, in Scientific and Clinical Applicationis of Magnetic Carriers, Hafeli et al, ed., Plenum Press, New York (1997); Lubbe and Bergemann, “Selected Preclinical and First Clinical Experiences with Magnetically Targeted 4′-Epidoxorubicin in Patients with Advanced Solid Tumors”, in Scientific and Clinical Applications of Magnetic Carriers, Hafeli et al, ed., Plenum Press, New York (1997); Hongming and Langer, J. Pharm. Res. 14:537 (1997); Hafeli et al., J. Bioined. Mater. Res. 28:901-908 (1994); Muller-Schulte et al., “A new AIDS therapy approach using magnetoliposomes” in Scientific and Clinical Applications of Magnetic Carriers, Hafeli et al, ed., Plenum Press, New York, (1997)).

Such previously known compositions have not proven practical and/or effective. Often, there is ineffective drug concentration delivered to the targeted site. (Lubbe and Bergemann, “Selected Preclinical and First Clinical Experiences with Magnetically Targeted 4′-Epidoxorubicin in Patients with Advanced Solid Tumors” in Scientific and Clinical Applications of Magnetic Carriers, Hafeli et al, ed., Plenum Press, New York (1997)). Many of the compositions lack adequate transport capacity, exhibit weak magnetic susceptibility, and/or require extremely high flux density magnetic fields that are neither practical nor common to localize the particles. When these compositions are used, there is no real localization of the particles to provide a precise local therapy. Other shortcomings include non-specific binding and toxicity to untargeted organs due to the inefficient targeting. Some compositions are difficult to manufacture or prepare consistently, sterilize, and store without changing their designated properties.

Although many efforts have been made to develop and improve magnetite-based particles, no efforts have been made to incorporate magnetic components, such as, magnetic iron sulfides such as pyrrhotite (Fe₇S₈), and greigite (Fe₄S₄), magnetic ceramics such as Alnico 5, Alnico 5 DG, Sm₂Co₁₇, SmCo₅ and NdFeB, magnetic iron alloys, such as jacobsite (MnFe₂O₄), trevorite (NiFe₂O₄), awaruite (Ni₃Fe) and wairauite (CoFe), and magnetic metals such as metallic iron (Fe), cobalt (Co), nickel (Ni), and a biologically active agent and a polymeric material with appropriate particle size and distribution to achieve effective targeting and retention of the particles to the target area. For example, major difficulties have been experienced using metallic iron particles because they are not stable and are readily oxidized in the air. Indeed it is because of its tendency to oxidize, that metallic iron has not been used to prepare magnetically targetable polymeric particles. Moreover, the sensitivity of iron to oxidation renders any encapsulation process very challenging as oxidation of iron would transform iron to iron oxide, dramatically reducing the magnetic responsiveness of the final particles. In addition, the high density of metallic iron makes it very difficult to encapsulate the iron particles into polymer matrices using any suspension or emulsion techniques.

It is an object of the invention to provide a magnetically targetable composition comprising a magnetic material of different chemical and physical structure and a higher magnetic susceptibility than the previously reported magnetite. The instant invention meets this objective and also provides methods for producing such magnetically targetable compositions and addresses the above-enumerated deficiencies in the prior art.

### SUMMARY OF THE INVENTION

The present invention provides a magnetically responsive material that is incorporated into a particle, which further comprises a polymer and a biologically active agent The magnetic component has the general properties of having Curie temperatures (Tc) greater than the normal human body temperature (37° C.), having high magnetic saturation (>approximately 20 Am²/kg), and being ferromagnetic or ferrimagnetic. Examples of suitable magnetic components include magnetic iron sulfides such as pyrrhotite (Fe₇S₈), and greigite (Fe₄S₄), magnetic ceramics such as Alnico 5, Alnico 5 DG, Sm₂Co₁₇, SmCo₅ and NdFeB, magnetic iron alloys, such as jacobsite (MnFe₂O₄), trevorite (NiFe₂O₄), awaruite (Ni₃Fe) and waitauite (CoFe), and magnetic metals such as metallic iron (Fe), cobalt (Co), nickel (Ni). Each of the magnetic components can have added to its chemical formula specific impurities that may or not alter the magnetic properties of the material. Doped ferromagnetic or ferromagnetic materials within the above limits of Curie temperatures and magnetic saturation values are considered to be within the scope of the instant invention.

It is an object of this invention to provide a magnetically responsive composition of approximately 0.1 to approximately 30 μm in diameter, comprising about 1% to about 70% of polymer, about 30% to about 99% of magnetic component by mass, and a biologically active agent of about one part-per-billion to about 25% by mass. Preferably, the biologically active agent is selected for efficacy in diagnosing and/or treating a particular disease.

Another aspect of the invention is to provide a method for using a magnet to site-specifically target the particles of this invention for localized in vivo diagnosis or treatment of diseases.

Another aspect of this invention is a magnetically targetable particle comprising:
a) a magnetic component, wherein the magnetic component is not magnetite, hematite, or maghemite;
b) a biocompatible polymer; and
c) a biologically active agent

Another aspect of the this invention is to provide a method for producing magnetically targetable particles comprising combining the components:
a) a magnetic component, wherein the magnetic component is not magnetite, hematite, or maghemite;
b) a biocompatible polymer; and
c) a biologically active agent

Yet another aspect of the invention is a kit for administering a biologically active agent to a patient comprising a unit dose of magnetically targetable particles described above, and a vehicle enabling the administration of the particles.

Still another aspect of the invention is a method of sterilizing the magnetically targetable particles described above comprising irradiating the particles with a sterilizing amount of gamma irradiation.

Yet another aspect of the invention is a method for the localized in vivo delivery of a biologically active agent comprising:
a) suspending a magnetically targetable particle of this invention in a vehicle for injection;
b) injecting the vehicle loaded with the biologically active agent into a patient; and
c) establishing a magnetic field of sufficient strength to guide and retain a portion of the magnetically targetable particles at a site of interest.

### BRIEF DESCRIPTION OF THE DRAWINGS

FIG. 1 illustrates the particle size and size distribution for PLGA/Fe/CDDP using a light scattering technique.

FIG. 2 is the scanning electron micrograph of PLGA/Fe/CDDP particle, BMP-036/77 (A and B).

FIG. 3 is the magnetic saturation versus magnetic component content in particles.

FIG. 4 illustrates the magnetiziation curves of Bang's magnetite particles (NCOSN) vs. metallic iron-based particles.

FIG. 5 illustrates the magnetic capture of magnetic particles in an in vitro experimental system.

FIG. 6 illustrates the in vitro cell toxicity of PLGA/Fe microsphere without any drug in saline after 1 hr and 7 day degradation.

FIG. 7 is the in vitro cell cytotoxicity of CDDP released from PLGA/Fe/CDDP particles in suspension in saline (BMP-054-004).

FIG. 8 is the in vitro cell cytotoxicity of Poloxamer 407 alone on H460 cell line.

FIGS. 9A and B are scanning electron micrographs of PLGA/Fe/CDDP particles. The particles are shown at 1,000 times magnification is FIG. 9A and at 5,000 times magnification in FIG. 9B.

### DETAILED DESCRIPTION OF THE INVENTION

The present invention is a magnetically targetable composition comprising 1% to 70% of a biocompatible polymer, 30% to 99% of a magnetic component, and from one part-per-billion to about 25% of a biologically active agent by mass. With compositions having less than 1% polymer, the physical integrity of the particle is less than optimal. With compositions of greater than 70% polymer, the magnetic susceptibility of the particle is generally reduced beyond an optimal level for targeting biologically active substances in vivo. The compositions may be of any shape, different shapes conferring differing advantaegeous properties, with an average size of approximately 0.1 to approximately 30 μm in diameter.

The magnetic component has the general properties of having Curie temperatures (Tc) greater than the normal human body temperature (37° C.), having high magnetic saturation (>approximately 20 Am²/kg), and being ferromagnetic or ferrimagnetic. Examples of suitable magnetic components include magnetic iron sulfides such as pyrrhotite (Fe₇S₈), and greigite (Fe₄S₄), magnetic ceramics such as Alnico 5, Alnico 5 DG, Sm₂Co₁₇, SmCo₅ and NdFeB, magnetic iron alloys, such as jacobsite (MnFe₂O₄), trevorite (NiFe₂O₄), awaruite (Ni₃Fe) and wairauite (CoFe), and magnetic metals such as metallic iron (Fe), cobalt (Co), nickel (Ni). Each of the magnetic components can have added to its chemical formula specific impurities that may or may not alter the magnetic properties of the material. Doped ferromagnetic or ferrimagentic materials within the above limits of Curie temperatures and magnetic saturation values are considered to be within the scope of the instant invention. Specifically excluded from the magnetic components and the magnetically susceptible compositions of the instant invention are the iron oxides magnetite (Fe₃O₄), hematite (αFe₂O₃), and maghemite (γFe₂O₃).

The term “metallic iron” indicates that iron is primarily in its “zero valence” state (Fe⁰). Generally the metallic iron is greater than about 85% Fe⁰, and preferably greater than about 90% Fe⁰. More preferably the metallic iron is greater than about 95% “zero valence” iron. Metallic iron is a material with high magnetic saturation and density (218 emu/g and 7.8 g/cm³) which are much higher than magnetite (92 emu/g and 5.0 g/cm³). The density of metallic iron is 7.8 g/cm³, while magnetite is about 5.0 g/cm³. Thus, the magnetic saturation of metallic iron is about 4 fold higher than that of magnetite per unit volume. (CRC Handbook, 77th edition, CRC Press (1996-1997) and Craik, D., Magnetisn Principles and Applications, Wiley and Sons (1995).

The use of the magnetic components of this invention results in magnetically responsive compositions with significantly higher magnetic saturation (>50 emu/g). The higher magnetic saturation allows the carrier with biologically active agents to be effectively targeted to the desired site and eventually be extravasated through the blood vessel wall to enter into tissues.

The term “biocompatible polymer” is meant to include any synthetic and/or natural polymer that can be used in vivo. The biocompatible polymer may be bioinert and/or biodegradable. Some non-limiting examples of biocompatible polymers are polylactides, polyglycolides, polycaprolactones, polydioxanones, polycarbonates, polyhydroxybutyrates, polyalkylene oxalates, polyanhydrides, polyamides, polyacrylic acid, poloxamers, polyesteramides, polyurethanes, polyacetals, polyorthocarbonates, polyphosphazenes, polyhydroxyvalerates, polyalkylene succinates, poly(malic acid), poly(amino acids), alginate, agarose, chitin, chitosan, gelatin, collagen, atelocollagen, dextran, proteins, and polyorthoesters, and copolymers, terpolymers and combinations and mixtures thereof.

The biocompatible polymers can be prepared in the form of matrices. Matrices are polymeric networks. One type of polymeric matrix is a hydrogel, which can be defined as a water-containing polymeric network. The polymers used to prepare hydrogels can be based on a variety of monomer types, such as those based on methacrylic and acrylic ester monomers, acrylamide (methacrylamide) monomers, and N-vinyl-2-pyrrolidone. Hydrogels can also be based on polymers such as starch, ethylene glycol, hyaluran, chitose, and/or cellulose. To form a hydrogel, monomers are typically crosslinked with crosslinking agents such as ethylene dimethacrylate, N,N-methylenediacrylamide, methylenebis(4-phenyl isocyanate), epichlarohydin glutaraldehyde, ethylene dimethacrylate, divinylbenzene, and allyl methacrylate. Hydrogels can also be based on polymers such as starch, ethylene glycol, hyaluran, chitose, and/or cellulose. In addition, hydrogels can be formed from a mixture of monomers and polymers.

Another type of polymeric network can be formed from more hydrophobic monomers and/or macromers. Matrices formed from these materials generally exclude water. Polymers used to prepare hydrophobic matrices can be based on a variety of monomer types such as alkyl acrylates and methacrylates, and polyester-forming monomers such as ε-caprolactone, glycolide, lactic acid, glycolic acid, and lactide. When formulated for use in an aqueous environment, these materials do not need to be crosslinked, but they can be crosslinked with standard agents such as divinyl benzene. Hydrophobic matrices can also be formed from reactions of macromers bearing the appropriate reactive groups such as the reaction of diisocyanate macromers with dihydroxy macromers, and the reaction of diepoxy-containing macromers with dianhydride or diamine-containing macromers.

The biocompatible polymers can be prepared in the form of dendrimers. The size, shape and properties of these dendrimers can be molecularly tailored to meet specialized end uses, such as a means for the delivery of high concentrations of carried material per unit of polymer, controlled delivery, targeted delivery and/or multiple species delivery or use. The dendrimeric polymers can be prepared according to methods known in the art, for example, U.S. Pat. No. 4,587,329 or 5,714,166. Polyamine dendrimers may be prepared by reacting ammonia or an amine having a plurality of primary amine groups with N-substituted aziridine, such as N-tosyl or N-mesyl aziridine, to form a protected first generation polysulfonamide. The first generation polysulfonamide is then activated with acid, such as sulfuric, hydrochloric, trifluoroacetic, fluorosulfonic or chlorosulfonic acid, to form the first generation polyamine salt. The first generation polyamine salt can then be reacted further with N-protected aziridine to form the protected second generation polysulfonamide. The sequence can be repeated to produce higher generation polyamines. Polyamidoamines can be prepared by first reacting ammonia with methyl acrylate. The resulting compound is reacted with excess ethylenediamine to form a first generation adduct having three amidoamine moieties. This first generation adduct is then reacted with excess methyl acrylate to form a second generation adduct having terminal methyl ester moieties. The second generation adduct is then reacted with excess ethylenediamine to produce a polyamidoamine dendrimer having ordered, second generation dendritic branches with terminal amine moieties. Similar dendrimers containing amidoamine moieties can be made by using organic amines as the core compound, e.g., ethylenediamine which produces a tetra-branched dendrimer or diethylenetriamine which produces a penta-branched dendrimer.

The biocompatible polymers of this invention may be, for example, biodegradable, bioresorbable, bioinert, and/or biostable. Bioresorbable hydrogel-forming polymers are generally naturally occurring polymers such as polysaccharides, examples of which include, but are not limited to, hyaluronic acid, starch, dextran, heparin, and chitosan; and proteins (and other polyamino acids), examples of which include but are not limited to gelatin, collagen, fibronectin, laminin, albumin and active peptide domains thereof. Matrices formed from these materials degrade under physiological conditions, generally via enzyme-mediated hydrolysis.

Bioresorbable matrix-forming polymers are generally synthetic polymers prepared via condensation polymerization of one or more monomers. Matrix-forming polymers of this type include polylactide (PLA), polyglycolide (PGA), polylactide coglycolide (PLGA), polycaprolactone (PCL), as well as copolymers of these materials, polyanhydrides, and polyortho esters.

Biostable or bioinert hydrogel matrix-forming polymers are generally synthetic or naturally occurring polymers which are soluble in water, matrices of which are hydrogels or water-containing gels. Examples of this type of polymer include polyvinylpyrrolidone (PVP), polyethylene glycol (PEG), polyethylene oxide (PEO), polyacrylamide (PAA), polyvinyl alcohol (PVA), and the like.

Biostable or bioinert matrix-forming polymers are generally synthetic polymers formed from hydrophobic monomers such as methyl methacrylate, butyl methacrylate, dimethyl siloxanes, and the like. These polymer materials generally do not possess significant water solubility but can be formulated as neat liquids which form strong matrices upon activation. It is also possible to synthesize polymers which contain both hydrophilic and hydrophobic monomers.

The polymers of the invention can optionally provide a number of desirable functions or attributes. The polymers can be provided with water soluble regions, biodegradable regions, hydrophobic regions, as well as polymerizable regions.

Methods for forming various polymers and matrices are well know in the art For example, various methods and materials are described in U.S. Pat. No. 6,410,044; PCT Publication No. WO 93/16687; U.S. Pat. No. 5,698,213; U.S. Pat. No. 6,312,679; U.S. Pat. No. 5,410,016; and U.S. Pat. No. 5,529,914, U.S. Pat. No. 5,501,863 which are all incorporated herein by reference.

The methods used to produce the particles result in particles that comprise one or more magnetic components, one or more biocompatible polymers and one or more biologically active agents. Unlike previous compositions, the amount of iron oxide in the compositions of the present invention is limited and thus is present in a very small amount if there is any, for example, less than 5%. The magnetic components of the present invention are well-known materials with high magnetic susceptibility. Many of the magnetic components are commercially available in a variety of grades, including pharmaceutical grade.

Before the preparation of the particles, the magnetic component can be processed to have a different shape, size, surface area, and surface chemistry to improve the compatibility with the polymer, biologically active agent, or incorporation efficiency. Many different processes can be used to increase and to optimize the compatibility with polymers and magnetic susceptibility of the magnetic component and to improve the incorporation efficiency. For example, raw magnetic material can undergo gas phase treatment or activation, milling, thermal activation, chemical vapor deposition of functional groups or any other of a variety of techniques apparent to any person skilled in the art (See, e.g., Reynoldson, R. W. Heat Treatment of Metals, 28:15-20 (2001); Ucisik et al., J. Australasian Ceramic Soc., 37, (2001); Isaki et al., Japanese Patent 08320100 (1996); and Pantelis et al., “Large scale pulsed laser surface treatment of a lamellar graphite cast iron”, Surface Modification Technologies VIII. Proceedings, 8^{th} International Conference, Nice, France, 26-28 Sep. 1994, eds. T. S. Sudarshan, M. Jeandin, J. 3. Stiglich, W. Reitz. Publ: London SWIY 5 DB, UK The Institute of Materials, 297-309 (1995)).

The high-energy milling process consists of combining the magnetic powder with a liquid, for example ethanol, in a canister containing grinding balls. The liquid serves as a lubricant during the milling process and also inhibits the oxidation of the powder; an especially important consideration when fabricating magnetic particles comprising iron. The canisters are then placed in a laboratory planetary mill of the type characteristically used in metallurgy (i.e., mill made by Fritsch, Germany). Other types of mills producing similar results may also be employed. The mill is ran for an appropriate time (generally between 1 and 10 hours) at speeds, for example, between 100 and 1000 rpm. At the end of the cycle, the magnetic component is collected. The magnetic component may be re-suspended and homogenized if desired. The magnetic component may be dried by any suitable technique, allowing for the protection of the material against oxidation. This process results in elongation of material, rendering it more magnetically susceptible due to increased pole separation, with larger surface area per mass of magnetic substance.

Another process includes subjecting the magnetic component to a gas phase treatment. For example, the magnetic component may be placed in a quartz container within an oven. Hydrogen may be used to replace air in the oven and the temperature is then raised for example, to about 300° C. The magnetic component is left in this environment for about 2 hours. At the end of the cycle, the temperature is lowered and hydrogen is replaced by nitrogen. Once the magnetic component's temperature has been returned to room temperature, it is collected and packaged. This process results in an increase in the roughness of the magnetic component's surface, leading to enhanced attachment of the biocompatible polymer and the biologically active agent.

In one embodiment of the invention the magnetic component has a size of about 0.05 to about 30 microns, more preferably between 0.1 and 10 microns. Typically, any magnetic component is essentially chemically pure. For example, when metallic iron is used as the magnetic component its purity is higher than 85% metallic iron, more preferably higher than 90% metallic iron, and most preferably higher than 95% metallic iron. The magnetic components may be commercially available or further processed to obtain the desired size and surface properties.

The magnetically targetable particles can be prepared using various processes including, but not limited to, emulsion, solvent evaporation emulsion, suspension, coacervation, precipitation, spray drying, spray coating, and bubble drying. For example, in an emulsion process, the polymer is dissolved in a solvent Then the magnetic component is dispersed in the resulting solution. Various amounts of biologically active agent are dispersed in the resulting suspension. The mixture is then emulsified with or without a surfactant Homogenization can be continued until the desired average size and size distribution is obtained. The solvent can then be evaporated. Optionally, the particles can be washed with a solution or solvent. Collected particles may be dried, for example, under vacuum in a vacuum oven. Particles can be stored at room temperature or low temperature.

One or more biologically active agents are incorporated with the particles for delivery to specific sites under control of a magnetic field. A biologically active agent can be incorporated with the particle by a linkage. For example, a biologically active agent can be covalently linked to the polymer, either directly or through a linker. Alternatively, a biologically active agent can be ionically linked, or associated, to the polymer, either directly or through a linker or a derivative. The biocompatible polymer can also be contained within a polymer matrix, such as a hydrogel or a block copolymer, and permitted to diffuse from the particle at a controlled rate. The rate of diffusion of the biologically active agent can be controlled by varying the composition of the matrix.

The term “biologically active agent” is meant to include any material having diagnostic and/or therapeutic properties including, but not limited to, small molecules, macromolecules, peptides, proteins, enzymes, DNA, RNA, genes, cells, or radionuclides. Non-limiting examples of therapeutic properties are antimetabolite, antifungal, anti-inflammatory, antitumoral, antiinfectious, antibiotic, nutrient, agonists, and antagonist properties. The terms “a” and “one” are both meant to be interpreted as “one or more” and “at least one.”

The term “biologically active agent” also includes compounds used for diagnostic purposes and having no apparent physiological or therapeutic effect Bifunctional agents having both diagnostic and therapeutic properties are also contemplated.

Non-limiting examples of biologically active agents include antineoplastics, blood products, biological response modifiers, anti-fungals, antibiotics, hormones, vitamins, proteins, peptides, enzymes, dyes, anti-allergies, anti-coagulants, circulatory agents, metabolic potentiators, antituberculars, antivirals, antianginals, anti-inflammatories, antiprotozoans, antirheumatics, narcotics, opiates, diagnostic imaging agents, cardiac glycosides, neuromuscular blockers, sedatives, anesthetics, paramagnetic particles and radioactive molecules or particles.

More specifically, biologically active agents that can be incorporated with the magnetically targetable particles are, for example, but not limited to muscarinic receptor agonists and antagonists; anticholinesterase agents; catecholamines, sympathomimetic drugs, and andrenergic receptor antagonists; serotonin receptor agonists and antagonists; local and general anesthetics; anti-migraine agents such as ergotamine, caffeine, sumatriptan and the like; anti-epileptic agents; agents for the treatment of central nervous system degenerative disorders; opiod analgesics and antagonists; anti-inflammatory agents, including anti-asthmatic drugs; histamine and bradykinin antagonists, lipid-derived autocoids; nonsteroidal anti-inflammatory agents and anti-gout agents; anti-diuretics such as vassopressin peptides; inhibitors of the renin-angiotensin system such as angiotensin converting enzyme inhibitors; agents used in the treatment of myocardial ischemia, such as organic nitrates, Ca²⁺ channel antagnonists, beta-adrenergic receptor antagonists, and antiplateletlantithrombotic agents; anti-hypertensive agents such as diureitcs, vasodilators, Ca²⁺ channel antagnonists, beta-adrenergic receptor antagonists; cardiac glycosides such as digoxin, phosphodiesterase inhibitors; antiarrhythmic agents; anti-hyperlipoprotenimia agents; agents for the control of gastric acidity and treatment of peptic ulcers; agents affecting gastrointestinal water flux and motility; agents that cause contraction or relaxation of the uterus; anti-protozoal agents; anthelmintic agents; antimicrobial agents such as sulfonamides, quinolines, trimethoprim-sulfamethoxazole; beta-lactam antibiotics; aminoglycosides; tetracyclines; erythromycin and its derivatives; chloramphenicol, agents used in the chemotherapy of tuberculosis; Mycobacterium avium complex disease, and leprosy, anti-fungal agents; and anti-viral agents; anti-neoplastic agents such as alkylating agents, antimetabolites; natural products such as the vinca alkaloids, antibiotics (e.g., doxorubicin, bleomycin and the like); enzymes (e.g. L-asparaginase), biological response modifiers (such as interferon-alpha); platinum coordination compounds, anthracenedione and other miscellaneous agents; as well as hormones and antagonists (such as the estrogens, progestins, and the adrenocorticosteriods) and antibodies; immunomodulators including both immunosuppressive agents as well as immunostimulants; hematopoietic growth factors, anticoagulant, thrombolytic and antiplatelet agents; thyroid hormone, anti-thyroid agents, androgen receptor antagonists; adrenocortical steroids, insulin, oral hypoglycemic agents, agents affecting calcification and bone turnover as well as other therapeutic and diagnostic hormones, vitamins, minerals blood products biological response modifiers, diagnostic imaging agents, as well as paramagnetic and radioactive molecules or particles. Other biologically active substances may include, but are not limited to monoclonal or other antibodies, natural or synthetic genetic material and prodrugs.

As used herein, the term “genetic material” refers generally to nucleotides and polynucleotides, including nucleic acids, RNA and DNA of either natural or synthetic origin, including recombinant, sense and antisense RNA and DNA. Types of genetic material may include, for example, genes carried on expression vectors, such as plasmids, phagemids, cosmids, yeast artificial chromosomes, and defective (helper) viruses, antisense nucleic acids, both single and double stranded RNA and DNA and analogs thereof, as well as other proteins or polymers.

The biologically active agents for the targetable particles may also be radioisotopes. Such radioisotopes are chemical compounds or elements that emit alpha, beta or gamma radiation and that are useful for diagnostic and/or therapeutic purposes. One factor used in selecting an appropriate radioisotope is that the half-life be long enough so that it is still detectable or therapeutic at the time of maximum uptake by the target, but short enough so that deleterious radiation with respect to the host is minimized. Selection of an appropriate radioisotope would be readily apparent to one having ordinary skill in the art. Generally, alpha and beta radiation are considered useful for local therapy. Examples of useful therapeutic compounds include, but are not limited to ³²P, ¹⁸⁶Re, ¹⁸⁸Re, ¹²³I, ¹²⁵I, ¹³¹I, ⁹⁰Y, ¹⁶⁶Ho, ¹⁵³Sm, ¹⁴²Pr, ¹⁴³Pr, ¹⁴⁹Tb, ¹⁶¹Tb, ¹¹¹In, ⁷⁷Br, ²¹²Bi, ²¹³Bi, ²²³Ra, ²¹⁰Po, ¹⁹⁵Pt, ^{195m}Pt ²⁵⁵Fm, ¹⁶⁵Dy, ¹⁰⁹Pd, ¹²¹Sn, ¹²⁷Te, ¹⁰³Pd, ¹⁷⁷Lu, and ²¹¹At. The radioisotope generally exists as a radical within a salt, although exceptions such as iodine and radium exist wherein the radical is not in ionic form. Useful diagnostic radioisotopes exist, and are well-known to those ordinarily skilled in the art. The useful diagnostic and therapeutic radioisotopes may be used alone or in combination.

As a general principle, the amount of any biologically active agent incorporated can be adjusted by varying the proportion of magnetic component, polymer and biologically active substance at the start of the particle preparation process up to a maximum of about 25% by mass of the composite particles without loss of utility of the particles in the therapeutic treatment regimens described in this application. In many cases it has been observed that an increase in the amount of incorporated biologically active substance is approximately proportional to the increase in polymer content However, as both polymer and biologically active substance contents increase, the susceptibility, or responsiveness, of composite particles to a magnetic field decreases. Therefore, it is necessary to achieve a balance in the magnetic component:polymer:biologically active agent ratio to maintain the balance between targeting efficiency linked to the magnetic susceptibility or magnetic component content, and the therapeutic outcome linked to the agent loading. Appropriate ratios may be determined by any person having average skill in the art.

It has been determined that the useful range of magnetic component content for particles intended for use in in vivo therapeutic treatments is, as a general rule, from about 30% to about 99%. The maximum amount of the biologically active agent that can be incorporated in the magnetically targetable particle of any given magnetic component content will also differ depending upon the chemical nature of the biologically active agent. Any person having ordinary skill in the art will be able to determine the proper ratio for the desired application.

The magnetically targetable particle can be associated with other molecules or compounds for use in analytical or pharmaceutical applications. The combination of a magnetically targetable particle and another molecule or compound may be referred to as a “conjugate.” For example, the term “immunoconjugate” can refer to a conjugate comprising an antibody or antibody fragment and a magnetically targetable particle. Conjugates of a magnetically targetable particle and other molecules such as a label compound (e.g., a fluorophore), a binding ligand (e.g., a protein derivative), or a therapeutic agent (e.g., a therapeutic protein, toxin or organic molecule) can also be made by methods known in the art

Conjugates can be prepared by covalently coupling one of the conjugate components to the other. Often coupling involves the use of a linker compound or a molecule that serves to join the conjugate components. A linker is typically chosen to provide a stable coupling between the two components. The greater the stability of the linkage between the components of a conjugate, the more useful and effective the conjugate. Depending upon a conjugate's use, a wide variety of conjugates may be prepared by coupling one conjugate component to another via a linker.

Alternatively, chelating structures can be employed to maintain the association of radionuclides to the magnetically targetable particles. Useful chelating structures include diethyltriaminepentaacetic acid (DTPA), structures based on the diamidodithiol (DADT) and triamidomonothiol (TAMT) backbones, and phosphinimine ligands. (See, e.g., U.S. Pat. No. 5,601,800).

Additional targeting mechanisms can be optionally associated with the magnetically targetable particles. For example, an antibody, or fragment thereof, recognizing a specific ligand can be attached to the particles. Such immunoconjugates allow the selective delivery of biologically active agents to tumor cells. (See, e.g., Hermentin and Seiler, Behringer Insti. Mitl. 82:197-215 (1988); Gallego et al., Int. J. Cancer 33:773744 (1984); Amon et al., Immunological Rev. 62:5-27 (1982)). For example, an antibody or antibody fragment recognizing a tumor antigen can be attached to a magnetically targetable particle. The antibody-containing particle can then be located at a tumor site by both a magnetic field and by antibody-ligand interactions.

Antibodies and antibody fragments, including monoclonal antibodies, anti-idiotype antibodies, and Fab, Fab′, F(ab′)₂ fragments or any other antibody fragments, that recognize a selected antigen can be obtained by screening antibodies and selecting those with high affinity. (See, generally, U.S. Pat. Nos. RF 32,011, 4,902,614, 4,543,439 and 4,411,993; see also, Monoclonal Antibodies, Hybridonzas: A New Dimension in Biological Analyses, Plenum Press, Kennett, McKeam, and Bechtol (eds.), 1980; Antibodies: A Laboratory Manual, Harlow and Lane (eds.), Cold Spring Harbor Laboratory Press, 1988)). Alternatively, antibodies or antibody fragments may also be produced and selected utilizing recombinant techniques. (See, e.g., Huse et al., Science 246:1275-1281 (1989); see also, Sastry et al., Proc. Natl. Acad. Sci USA 86:5728-5732 (1989); Alting-Mees et al, Strategies in Molecular Biology 3:1-9 (1990)).

In addition, ligands recognized by receptors can be associated with a particle. For example, neuraminic acid or sialyl Lewis X can be attached to a magnetically targetable particle. Such a ligand-containing particle can then be located at a specific site, such as an endothelial site, by both a magnetic field and by ligand-selectin interactions. Such conjugates are suitable for the preparation of a medicament for treatment or prophylaxis of diseases in which bacterial or viral infections, inflammatory processes or metastasizing tumors are involved. Other ligands, such as protein or synthetic molecules that are recognized by receptors can be associated with a magnetically targetable particle. In addition, peptide, DNA and/or RNA recognition sequences can be associated with a magnetically targetable particle.

The association of the targeting mechanism can be by a covalent or ionic bond. U.S. Pat. No. 5,601,800 describes several methods for attaching biologically active agents, such as diagnostic agents, contrast agents, receptor agents, and radionuclides to particles. Useful linkers and methods of use are described in, for example, U.S. Pat. No. 5,824,805; U.S. Pat. No. 5,817,742; U.S. Pat. No. 6,339,060.

Because it is convenient to prepare and market the magnetically targetable particles in a dry form, the excipients may be prepared in dry form, and one or more dry excipients are packaged together with a unit dose of the magnetically targetable particles. A wide variety of excipients may be used, for example, to increase stability and biodegradability. The type and amount of appropriate dry excipients will be determined by one skilled in the art depending upon the chemical properties of the biologically active agent. The magnetic particles may be optionally washed, dried, recovered, sterilized and/or filtered. Routine methods of packaging and storing may be employed. For example, the raw or processed dried particles may be packaged in appropriate container closure system, for example, one enabling unit dosage forms. Packaging under nitrogen, argon or other inert gas is preferred to limit the oxidation of the particles. Although the particles may be stored “wet,” the liquid should not be aqueous. For example, ethanol or DMSO may be employed. (See, e.g., Kibbe, AH, Handbook of Pharmaceutical Excipients, American Pharmaceutical Association, Washington, D.C. (2000)).

A wide variety of excipients may be used, for example, to enhance precipitation or release of the biologically active agent. The type and amount of appropriate dry excipients can readily be determined by any person having ordinary skill in the art. For instance, the excipients can be selected from a viscosity agent or a tonicifier, or both. Viscosity agents are, for example, biodegradable polymers such as carboxymethylcellulose, PVP, polyethylene glycol (PEG), polyethylene oxide (PEO) and the like. Tonicifiers include sodium chloride, mannitol, dextrose, lactose, and other agents used to impart osmolarity to the reconstituted solution. Most preferably, the package or kit containing both the dry excipients and dry magnetically targetable particles are formulated to be mixed with the liquid contents of a vial containing a unit dose of the biologically active agents. Liquid agents could be used as excipients just prior to use of the particles. Such liquid agents could be soybean oil, rapeseed oil, or an aqueous based polymer solution comprising a polymer listed above. Also liquid solutions could be a tonicifier, such as Ringer's solution, 5% dextrose solution, and physiological saline. As before a combination of liquid excipients and tonicifiers can be used. (See, e.g., Kibbe, AH, Handbook of Pharmaceutical Excipients, American Pharmaceutical Association, Washington, D.C., 2000). Suitable delivery systems will be apparent to any person possessing ordinary skill in the art. Without limitation, examples of useful delivery systems include matrices, capsules, slabs, microspheres, and liposomes. Conventional excipients may be incorporated into any of the formulations.

A diagnostic or therapeutic amount of a biologically active agent associated with the magnetically targetable particles will be determined by one skilled in the art as that amount necessary to effect diagnosis or treatment of a particular disease or condition, taking into account a variety of factors such as the patient's weight, age, and general health, the diagnostic or therapeutic properties of the drug, and the nature and severity of the disease. For example, the amount of particles administered to a patient constitutes a unit dose of the biologically active agent. The amount can be reduced in light of the efficiency of the delivery of the agent to the disease site in the patient due to the magnetic targeting properties of the particles. However, in one aspect of the present invention the maximum content of biologically active agent in a particle is 25% by weight.

A number of considerations are involved in determining the size of carrier particles to be used for any specific therapeutic situation. For particles less than about 0.1 tom in size, the magnetic control in blood flow and the carrying capacity is reduced. Relatively large particle sizes can tend to cause embolization of blood vessels during injection either mechanically or by facilitating clot formation by physiological mechanisms. Embolization of blood vessels is desirable or undesirable, depending on the circumstances. A dispersion may coagulate, which makes injections more difficult, and the rate at which biologically active substances release from the particles in the targeted pathological zones may decrease. The method (such as is described below) of coating magnetically targetable particles or incorporating a magnetic component and a biologically active substance into polymeric matrix produces an irregularly or spherically shaped form, and results in a particle population having an average major dimension of about 0.1 μm to about 10 μm.

The magnetically targetable particles are such that the biologically active agent can be associated with the particle, e.g., adsorbed, grafted, encapsulated, or linked to the particle. The content of biologically active agent in the final particle is between about one part-per-billion to about 25% of the final particle mass. As used herein, “associated with” means that the biologically active agent can be physically encapsulated or entrapped within the particle, dispersed partially or fully throughout the particle, or attached or linked to the particle or any combination thereof, whereby the attachment or linkage is by means of covalent bonding, hydrogen bonding, adsorption, absorption chelation, metallic bonding, van der Walls forces or ionic bonding, or any combination thereof. The association of the biologically active agent(s) and the particles(s) may optionally employ connectors and/or spacers to facilitate the preparation or use of the conjugates. Suitable connecting groups are groups which link a biologically active agent to the particle without significantly impairing the effectiveness of the biologically active agent or the effectiveness of any other carried material present in the particle. These connecting groups may be cleavable or non-cleavable and are typically used in order to avoid steric hindrance between the biologically active agent and the particle. Since the size, shape and functional group density of the particle can be rigorously controlled, there are many wvays in which the biologically active agent can be associated with the particle. For example, (a) there can be covalent, coulombic, hydrophobic, or chelation type association between the biologically active agent(s) and entities, typically functional groups, located at or near the surface of the particle; (b) there can be covalent, coulombic, hydrophobic, or chelation type association between the biologically active agent(s) and moieties located within the interior of the particle; (c) the particle can be prepared to have an interior which is predominantly hollow allowing for physical entrapment of the biologically active agent within the interior (void volume), wherein the release of the biologically active agent can optionally be controlled by congesting the surface of the particle with diffusion controlling moieties, or (d) various combinations of the aforementioned phenomena can be employed.

The methods of use include methods for localized in vivo diagnosis and/or treatment of disease providing a magnetically targetable particle having incorporated thereon one or more biologically active agents selected for efficacy in diagnosing and/or treating the disease, and administrating the particle into the body of a patient in a variety of routes, including intra-arterial, intra-venous, intra-tumoral, intra-peritoneal, subcutaneous, etc. For example, but without limitations, the particles are injected by intra-arterially administration into an artery within a short distance from a body site to be treated and at a branch or branches, preferably the most immediate, to a network of arteries carrying blood to the site. The particles are injected through the delivery means (e.g., a needle or catheter) into the blood vessel. In one embodiment, prior to injection a magnetic field is established at a target site having sufficient field strength to guide a portion of the injected particles to, and retain a portion of the particles at the site. In another embodiment, the magnetic field is of sufficient strength to draw the particles into the soft tissue at the site adjacent to the network of vessels, thus avoiding substantial embolization of any of the larger vessels by the carrier particles, should embolization be undesirable. Examples of magnets for use according to the invention are a DC electromagnet or permanent magnet of sufficient size and strength to produce 100 gauss of magnetic flux at the target site. For example, the magnets discussed in Mitchiner et al., U.S. Pat. No. 6,488,615, issued Dec. 3, 2002, are suitable for use with the instant invention. In the case where the biologically active agent(s) includes a diagnostic imaging agent, the imaging is performed while the particles are aggregated at the target site, and in some cases before and/or after. Imaging modalities and methods are well-known to any person having ordinary skill in the art.

Particles may be subaliquoted into dosage units, for example, between 50 and 500 mg per dose, and may be further overlayed with nitrogen, for example. Dosage units may be sealed, for example, with butyl rubber stoppers and aluminum crimps. Dosage units may then be sterilized by appropriate sterilization techniques, for example, gamma irradiation between 2.5 and 4.0 Mrads. Other sterilization techniques may also be used, for example, dry heat and electron beam sterilization.

All books, articles and patents referenced herein are fully incorporated by reference. The following Examples illustrate various aspects of the invention and are in no way intended to limit the scope of the invention.

### EXAMPLES

### Example 1

### Particles Prepared Using Solvent Evaporation Emulsion Process

A composite magnetic particle made of poly(lactic acid-co-glycolic acid) (PLGA), metallic iron, and cisplatin (CDDP) was prepared using the solvent evaporation emulsion process. One gram of PLGA was dissolved in 13.6 g of methylene chloride (DCM). One gram of iron and 0.5 g of cisplatin were then dispersed in the resulting solution by sonication for 30 minutes. The organic phase was then emulsified with homogenizer (at a speed of 11,000 rpm) in 400 ml of saline solution (0.9% w/v) containing 8 g of polyvinyl alcohol and 0.4 g of Tween®-80. Previously, this solution was saturated with cisplatin (0.1%, w/v), and the pH was adjusted to 2 by the addition of concentrated HC1. Homogenizing was continued until the DCM was completely evaporated. The system was protected from light The particles were washed four times with cold water, collected by centrifugation, and dried under vacuum at room temperature for 48 h and stored at 4° C.

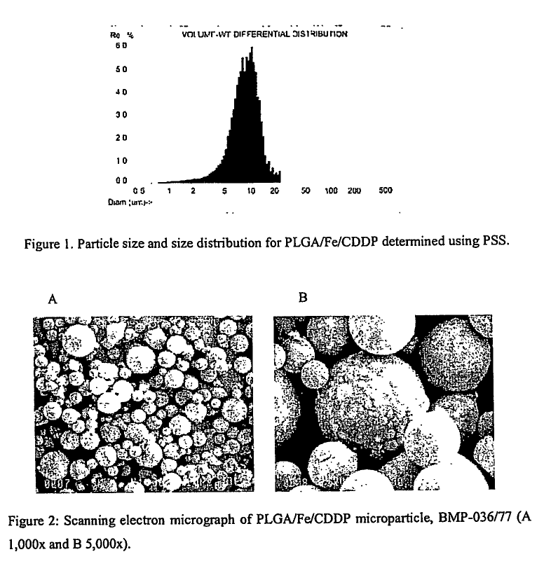

The size and size distribution of the polymer based magnetic microspheres were measured using light scattering (Accusizer 770A, Particle Sizing Systems, Santa Barbara, Calif.). FIG. 1 shows particle size and distribution for PLGA/Fe/CDDP particles. The number weighted average size of the particles was about 2.5 μm with a polydispersity of 3.6.

Morphology of the polymeric based magnetic particles was examined using scanning electron micrography (SEM) (Jeol-840, Jeol USA, Inc., Peabody, Mass.). The particles are spherical and iron particles are distributed through out the polymer particles, see FIG. 2.

The experimental determination of the contents of polymer, iron and cisplatin as determined by ICP-MS (Qualitative Technologies, Inc., Whitehouse, N.J.) and mass balance were 36.7%, 61.3% and 2% by weight respectively. The magnetic saturation of the magnetic particles was 93.3 emu/g.

### Example 2

### Particles Prepared Using Various Emulsifiers

Many emulsifiers such as PVA, Poloxamer 407 (P407), Poloxamer 188 (P-188), oleic acid/sodium hydroxide and Polysorbate-80 (Tween®-80) were investigated to stabilize microspheres during the emulsion process. Table 1 shows the effect of different emulsifiers on the size of particles as estimated by light microscopy (Wesco CXR3, Wesco, Burbank, CA). Poloxamer 188 at three different concentrations generated particles in the size range smaller than 10 μm. The organic/aqueous phase ratio did not change particle size significantly. When PVA and oleic acid were used as emulsifiers, the particle size was significantly smaller than when Poloxamer 188 was used. The particles generated from all of these experiments were free flowing spherical particles.

TABLE 1
Effect of emulsifiers on the size of particles

| Sample | | Fe/ | Organic/ | Particle |
|---|---|---|---|---|
| ID | Surfactant | PLGA | Aqueous | Size (μm) |
| 1 | 0.5% P-188 | 1/2 | 1/40 | 10 |
| 3 | 4% P-188 | 1/2 | 1/40 | 10 |
| 4 | 4% P-188 | 1/2 | 1/20 | 10 |
| 5 | 0.05% Oleic Acid | 1/2 | 1/20 | 2 |
| 6 | 2% PVA | 1/2 | 1/20 | 2 |
| 8 | 8% PVA | 1/2 | 1/20 | 2 |

The effect of charge and pH on the loadings of cisplatin and iron were investigated using PVA (2%) as emulsifier. The content of CDDP and metallic iron were analyzed with ICP-MS. The loading of CDDP at lower pH (2.5) was about 4% with about 57.3% of metallic iron. When the magnetic particles were prepared at a neutral pH, the loading of CDDP was about 2% with 61.3% of iron with a magnetic saturation of >90 emu/g. The loading of CDDP and metallic iron was increased with the increase of initial charge of CDDP.

### Example 3

### Particles Prepared Using Poloxamer 407 (P407) as an Emulsifier

The following examples provide a method for producing particles incorporating cisplatin and metallic iron in a poly(lactic acid-co-glycolic acid) (PLGA) matrix. Similar procedures can be used to with other magnetic components to provide the magnetically targetable compositions of this invention. A procedure similar to Example 1 was followed using emulsifier P407. The initial charge ratio of PLGA:Fe:CDDP was fixed at 1:0.5 for all of the experiments. The concentration of emulsifier P-407 was varied. The CDDP loading in the microspheres was about 15% (Table 2).

TABLE 2
Effect of emulsifiers on the size of particles

| | P-407 | CDDP | Average |
|---|---|---|---|
| Sample ID | (%) | Content (%) | Size (μm) |
| BMP54/004 | 2 | 15.4 | 0.99 |
| BMP54/006 | 4 | 15.2 | 0.94 |

### Example 4

### Magnetic Susceptibility

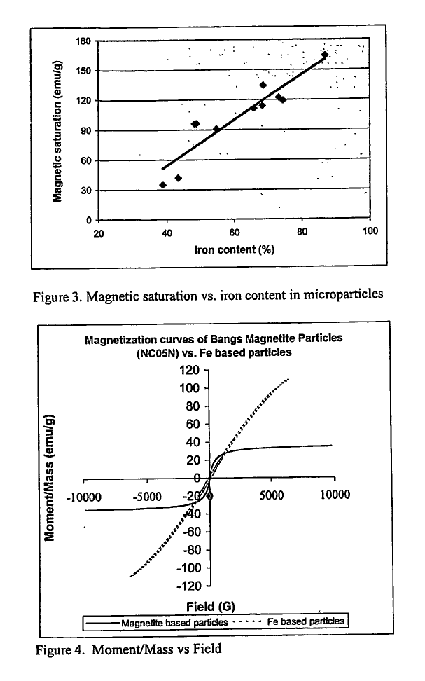

Example 4 contrasts the magnetic susceptibility of the metallic iron composite microparticles with those of magnetite based particles. Magnetic saturation vs. the iron content of these particles is shown in FIG. 3. The magnetic saturation increases with the iron content The greater the magnetic saturation, the greater the degree of the magnetic attraction (capture), and the deeper the particles can be targeted in vivo.

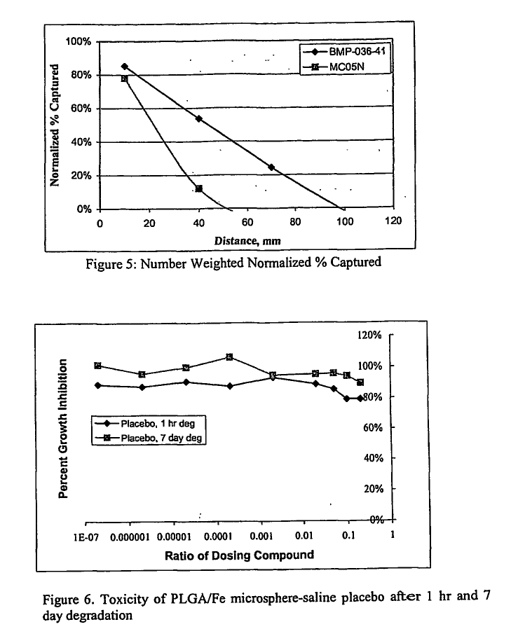

FIG. 4 illustrates the magnetiziation curves of Bang's magnetite particles (NC05N) vs. Fe based particles. The PLGA/Fe microparticles not only have a much higher magnetic saturation, they also have a different characteristic magnetization hysteresis curve. As shown in FIG. 5, a PLGA/Fe microparticle preparation (NB#036-21A) with 50.6% Fe has a magnetic saturation greater than 108 emu/g, while a generic magnetite based particle (Bangs Magnetite Particles, catalog MC05N, Poly(styrene-divinylbenzene 60/0N—COOH) Magnetite 52.4%, Inv. # L95121 ID, Bangs Lot# 1975), Bangs Laboratories, Inc., Fishers, Ind. has a saturation magnetization of only 34.7 emu/g. The theoretical saturation magnetization for magnetite and metallic iron are 92 and 218 emu/g, respectively (Craik, D., Magnetism Principles and Applications, Wiley and Sons, 1995). The labeled magnetite content of the particles is 52.4%, so a saturation magnetization of approximately 50 emu/g was expected. This shows that only 70% of the expected magnetic properties are retained by magnetite when it is dispersed as a fine powder and covered by polymer. In a like manner, the metallic iron-based particle, which is 50.6% Fe by weight, would be expected to have a saturation of 109 emu/g. Therefore, the metallic iron based particle retains approximately 100% of the expected magnetic saturation. This shows that while both particle types retain their magnetic properties, the metallic iron-based particle is better at retaining these properties when formed into a finely dispersed microsphere, and is unexpectedly superior to an iron oxide-based particle in terms of its magnetic properties.

### Example 5

### Magnetic Capture

This example demonstrates the importance of using metallic iron instead of iron oxide to achieve efficient magnetic capture and targeting. A microsphere comprising about 50% metallic iron was investigated for its capture by a magnetic field in a flow field. Some commercially available magnetic particles (MC05N, ˜1 μm in size and 60% of magnetite by weight from Bangs Laboratories) were used as reference. FIG. 5 illustrates the percent captured based on the number of particles vs. distance between the magnet and particles. The metallic iron-based microparticle, BMP-036-41, showed much higher magnetic capture efficiency. The magnetic capture for Bangs particles (MC05N) diminished quickly with the increase of distance from the magnet.

### Example 6

### In Vitro Evaluation of CDDP Release and Cytotoxicity

This example demonstrates that cisplatin encapsulated in the particles retains its biological activity when released from the particle. The cytotoxicity of CDDP released from microspheres was investigated using a non-small cell lung cancer cell line. The microspheres were suspended in saline solution and an aliquot was taken at different time points.

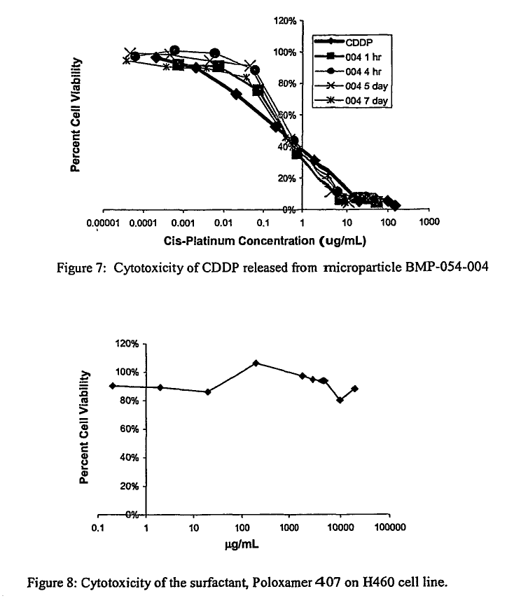

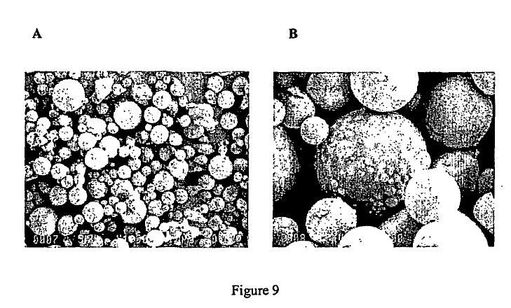

FIG. 7 shows cytotoxicity of CDDP released from the particle prepared in Example 3 after 1 hour, 4 hours, 5 days, and 7 days. The growth inhibition profile or activity of the drug released from particles is identical to the profile of CDDP that has not contacted particles. This indicates that the microsphere formation process did not change the cytotoxicity of CDDP. FIG. 6 shows that the drug free PLGA/Fe microspheres do not have any significant toxicity to the cell line tested.

The surfactant Poloxamer 407 used to generate PLGA/Fe/CDDP microspheres was also tested for its toxicity to the cell line. FIG. 8 shows the effect of Poloxamner 407 on cell viability. The highest possible equivalent concentration of polaxymer in any of the desorbed CDDP tested is 0.5 μg/mL, and this graph shows no excipient toxicity up to >10,000 μg/mL.

Overall, these results show that CDDP that has been encapsulated and released from magnetically targetable particles produces the same tumor growth inhibition effects in vitro as CDDP itself. The experimental IC₅₀ values are comparable to a published value of 0.537 μg/ml (Rixe, O.; Ortuzar, W.; Alvarez M.; Parker, R; Reed, E.; Paul, K.; Fojo, T.; Oxaliplatin, Tetraplatin, Cisplatin, and Carboplatin: Spectrum of activity in drug resistant cell lines and in the cell lines of the national Cancer Institute's Anticancer Drug Screen Panel; Biochem Pharmacol; 52; 1855-1865). Polymer microsphere excipient without drugs were not toxic. The cytotoxicity of CDDP was preserved in the solvent evaporation emulsion process.

### Example 7

### Particles Prepared Using Mixed Solvents as an Organic Phase

Example 7 describes a method for producing particles using a mixture of solvents as an oil inner phase. N,N-dimethylformamide (DMF), or dichloromethane (DCM), or a mixture of the two was used alternatively as the inner organic solvent and water containing one or two surfactants was used as the continuous phase. A selected amount of PLGA was dissolved in the solvent. CDDP and Fe powder were dispersed in the solution by sonication. The solution was then added to the continuous phase in different emulsification methods, such as intensive mechanical stirring, sonication, or homogenization. The emulsion obtained was agitated at 400-1000 rpm and the temperature of the emulsion was gradually raised to 40° C. The agitation was maintained at 40° C. for 2 h or 5 h under vacuum to evaporate the solvent The resulting suspension was centrifuged at 4400 rpm (5° C., 10 minutes) and the precipitate obtained was washed with hexane four times and then with 2-propanol twice. The particles were dried using a freeze-dry system for 24 h. When using a one-to-one or one-to-four ratio of DMF to DCM, the particle size was about 1-2 μm with relatively narrow size distribution.

### Example 8

### Particles Prepared by an Oil-in-Oil Emulsion and Solvent Evaporation Process

Example 8 describes a method for producing PLGA/CDDP/Fe particles using DCM or DMF as inner organic phase, and mineral oil containing 2% of lecithin as the continuous oil phase. The drug loading in the PLGA particles was determined using HPLC and elemental analysis and expressed as mg of CDDP per mg of particles. When using DCM as an inner organic solvent, CDDP was not soluble in both inner phase and continuous phase. The biologically active agent and metallic iron contents were about IP/o and 49% in the particles, respectively.

When DMF was used as the inner organic phase, the particles were prepared by using Span®-85 as emulsifier in the continuous oil phase. In this case, the biologically active agent loading was about 26% with about 40% metallic iron. The agent and metallic iron content in particles could be varied to have a suitable magnetic susceptibility and a therapeutic amount of CDDP.

### Example 9

### Preparation of Fe Gelatin Particles Using Coacervation Process

Example 9 describes a method for producing magnetically targetable particles containing carboplatin. The process involves adding an aqueous solution of gelatin containing the agent to an excess of dehydration solvent such as ethanol and cross-linking. Briefly, a flask containing 300 or 500 ml of dehydrating solvent was immersed in a dish containing dry ice in isopropanol in order to maintain the temperature at −15° C. Anhydrous ethanol and isopropanol were used as dehydrating solvents. The dehydrating solvent was mechanically stirred at a speed of 300 to 500 rpm or homogenized at a speed of 11,000 1/minutes. Then 5 or 10 mL of 0.5% (w/v) aqueous gelatin solution containing 400 mg of iron powder was added drop-wise into the flask. The stirring or homogenization was maintained at −15° C. for 15-30 minutes. The droplets were crosslinked by adding 25% (w/v) glutaraldehyde and stirred continuously for 45 minutes. The flask was then transferred to a refrigerator (about 4° C.) for 24 hours to 48 hours to allow the completion of the cross-linking process. Low temperature was used to keep the particles in the condensed form during the crosslinking. Resulting particle properties are shown in Table 3.

TABLE 3
Particles prepared by the gelatin coacervation process

| | | Particle size | |
|---|---|---|---|
| | Materials added in the | Number/volume | Fe content |
| Sample | preparation | mean size (μm) | (%, wt/wt) |
| Batch | Gelatin 50 mg/10 ml, Fe | 3.5/12.4 | 85.8 |
| 1002-4 | 400 mg | | |
| Batch | Gelatin 25 mg/5 ml, Fe | 1.4/47.9 | 95.3 |
| 1002-3 | 400 mg | | |

### Example 10

### Preparation of Carboplatin/Fe Gelatin Particles Using Emulsification Process

This process involves emulsifying an aqueous solution of gelatin in an oil phase and dehydrating with acetone. Briefly, 125 mg of metallic iron powder were dispersed by sonication in 5 ml of aqueous solution of gelatin with 50 mg of carboplatin. This suspension was slowly added to 50 g of an oil phase (castor oil, silicone oil, and mineral oil) previously heated to 80° C. In addition to the three different oils, in some cases the surfactants Tween 85 and Span 85 were used. The mixture was vigorously stirred (stirring speed varied from 900 to 12000 rpm) to form a w/o emulsion. After 30 minutes, the emulsion was rapidly cooled to 15° C., and then 30 ml of acetone was added to dehydrate the gelatin droplets.

During the particle preparation, 70 mL of glutaraldehyde (GTA) solution (25% w/v) was added) in some cases to cross-link the gelatin particles. Glutaraldehyde (GTA) was left to react for 24 hours. The particles were collected by centrifugation at 4400 rpm for 10 minutes at 5° C., washed with acetone four times, and then freeze-dried.

The gelatin particles incorporating carboplatin and metallic iron were prepared under the same conditions as iron-loaded gelatin particles. Several examples are shown in Table 4. The reproducibility is very good with the biologically active agent content ranging from 13.4% to 15.1% and metallic iron content ranging from 65.9% to 68.8%. Generally, the iron content and agent loading could be varied by changing the amount of agent, metallic iron, and gelatin used in the particle formation process. In another preparation, magnetically targetable particles with 12% carboplatin and 73.3% iron were obtained.

TABLE 4
Main characteristics of carboplatin-Fe-loaded gelatin particles

| | | | Agent | Fe |
|---|---|---|---|---|
| | | Particle | Loading | Content |
| | Materials added in the | Size | (%, | (%, |
| Sample | preparation | (μm) | wt/wt) | wt/wt) |
| Batch A | Gelatin 50 mg/5 ml, Fe 125 | 0.9 | 14.2 | 68.1 |
| | mg, carboplatin 50 mg | | | |
| Batch B | Gelatin 50 mg/5 ml, Fe 125 | 0.9 | 15.1 | 68.8 |
| | mg, carboplatin 50 mg | | | |
| Batch C | Gelatin 50 mg/5 ml, Fe | 0.9 | 13.4 | 65.9 |
| | 125 mg, carboplatin 50 mg | | | |

### Example 11

### Particles Prepared with Metallic Iron and PLGA-Lysozyme Conjugate

This example describes a method for producing magnetic targetable particles with PLGA-Lysozyme conjugate (Nam and Park, J. Microencapsulation 16:625-637 (1999) using the solvent evaporation emulsion technique. In this example the pharmaceutical agent (Lysozyme) is chemically linked to the polymer (PLGA). The PLGA-Lysozyme conjugate used in this example contains about 11% lysozyme. A 1 L reactor with a mechanical stirrer was charged with 300 mL of purified water, 2.7 g NaCl (0.9% w/v) and 3 g Poloxamer 188 (1% w/v). The contents were stirred until all the solids had dissolved. The clear solution was cooled in an ice-water bath. PLGA-Lysozyme conjugate (0.250 g) was dissolved in a co-solvent mixture of 1 mL DMSO and 1 mL methylene chloride. Iron powder (0.250 g) was dispersed in the organic solution by sonication for 5 minutes. The organic phase was then added drop-wise with stirring to aqueous phase, and the resulting mixture emulsified with a homogenizer for 0.5 hours. The resulting mixture was stirred at room temperature for 5 hours to extract the DMSO into the aqueous phase and evaporate the methylene chloride. The particles were collected by centrifugation, washed 2 times with 150 mL water, twice with 100 mL water, and lyophilized for 48 hours. The average metallic iron and lysozyme content in the particles were 45.2%±0.3% and 6.1%, respectively. The mean diameter of the particles was 3.3 μm.

### Example 12

### Particles Prepared with Metallic Iron and PLGA-DOTA Conjugate

This example describes a method for producing magnetically targetable particles comprising PLGA-DOTA using a solvent evaporation emulsion technique. DOTA or 1,4,7,10-tetraazacyclododecane-1,4,7,10-tetraacetic acid is a high affinity metal ion chelator that is useful in imaging and nuclear medicine. In this example, a conjugate to the polymer, DOTA, is available for conjugation to a biologically active substance, in this case through chelation. Furthermore, the biologically active substance, in this case a radionuclide for either diagnostic or therapeutic applications, would be present in trace amounts. A 1 L reactor with a mechanical stirrer is charged with 300 mL of purified water, 2.7 g NaCl (0.9% w/v) and 3 g Poloxamer 188 (1% w/v). The contents are stirred until all the solids dissolve. The clear solution is cooled in an ice-water bath. PLGA-DOTA conjugate (0.250 g) is dissolved in a co-solvent mixture of 1 mL DMF and 1 mL methylene chloride. Metallic iron powder (0.250 g) is dispersed in the organic solution by sonication for 5 minutes. The organic phase is then added drop-wise with stirring to the aqueous phase, and the resulting mixture emulsified with a homogenizer for 0.5 hours. The resulting mixture is stirred at room temperature for 5 hours to extract the DMF into the aqueous phase and evaporate the methylene chloride. The particles are collected by centrifugation, washed two times with 150 mL water, twice with 100 mL water, and lyophilized for 48 hours.

### Examples 13

### Magnetically Targetable Particles Encapsulating Mitomycin C

A composite magnetic particle made of poly(lactic acid-co-glycolic acid) (PLGA), metallic iron, and Mitomycin C (MMC) is prepared using a solvent evaporation emulsion process. The procedure of Example 1 was used. One gram of PLGA is dissolved in 13.6 g of methylene chloride (DCM) One gram of iron and 0.5 g of MMC are then dissolved in the resulting solution by sonication for 30 minutes. The organic phase is then emulsified with a homogenizer (at a speed of 11,000 rpm) in 400 ml of saline solution (0.9% w/v) containing 8 g of polyvinyl alcohol and 0.4 g of Tween-80. Homogenizing is continued until the DCM is completely evaporated. The system is protected from light The microspheres are washed four times with cold water, collected by centrifugation, and dried under vacuum at room temperature for 48 h and stored at 4° C.

Using the methodology described in Examples 1-3 or 7-13 detailed above, individual non-aggregated magnetically targetable particles are obtained similar to those shown in FIG. 2. They described in Examples 4. The magnetically targetable particles are free flowing, do not clump nor do they aggregate.

### Example 14

### Preparation of Oxaliplatin/Metallic Iron/Gelatin Particles Using Emulsification Process

This example describes a method for producing magnetically targetable particles incorporating oxaliplatin. The process involves emulsifying an aqueous solution of gelatin in the oil phase and dehydrating with acetone. Briefly, 400 mg of metallic iron power are dispersed by sonication in 5 ml of aqueous solution of gelatin with oxaliplatin. This suspension is slowly added to 50 g of an oil phase previously heated to 80° C. The mixture is vigorously stirred to form a w/o emulsion. After 30 minutes, the emulsion is rapidly cooled to 15° C., and then 30 ml of acetone is added to dehydrate the gelatin droplets.

During the particle preparation, 400 μL of glutaraldehyde (GTA) solution (4% w/v) is added to cross-link the gelatin particles. GTA is left to react for 24 h. The particles are collected by centrifugation at 4400 rpm for 10 min at 5° C., washed with acetone 4 times, and then freeze-dried.

Those skilled in the art will recognize that, while specific embodiments have been illustrated and described, various modifications and changes may be made without departing from the spirit and scope of the invention.

1-52. (canceled)

53. A magnetically targetable particle comprising;
a) a magnetic component;
b) a biocompatible polymer; and
c) a biologically active agent.

54. The particle of claim 53, wherein the magnetic component is selected from the group consisting of a magnetic iron sulfide, a magnetic ceramic, a magnetic iron alloy, and a magnetic metal.

55. The particle of claim 54, wherein the magnetic iron sulfide is selected from the group consisting of pyrrhotite and greigite, the magnetic ceramic is selected from the group consisting of Alnico 5, Alnico 5 DG, Sm2Co, 7, SmCos and NdFeB, the magnetic iron alloy is selected from the group consisting of jacobsite, trevorite, wairauite and awaruite, and the magnetic metal is selected from the group consisting of metallic iron, cobalt, and nickel.

56. The particle of claim 55, wherein the magnetic component is metallic iron.

57. The particle of claim 53, wherein the biocompatible polymer is selected from the group consisting of a hydrogel and a dendrimer.

58. The particle of claim 53, wherein the magnetic component comprises about 30% to about 99% by weight of said particle.

59. The particle of claim 53, wherein the one or more polymers comprise about 1% to about 70% by weight of said particle.

60. The particle of claim 53, wherein the biologically active agent, or agents comprise between about one part-per-billion and about 25% by weight of said particle.

61. The particle of claim 53, wherein the particle has a magnetic saturation greater than 50 emu/g.

62. The particle of claim 53, wherein said particle has a particle size range from about 0.1 to about 30 m.

63. The particle of claim 53, further comprising a pharmaceutically acceptable excipient.

64. The particle of claim 53, wherein the biologically active agent is associated with the biocompatible polymer by a biodegradable or hydrolyzable moiety.

65. The particle of claim 53, wherein the biocompatible polymer is selected from the group consisting of polylactides, polyglycolides, polylactide-coglycolide, polycaprolactones, polydioxanones, polycarbonates, polyhydroxybutyrates, polyalkylene oxalates, polyanhydrides, polyamides, polyacrylic acid, poloxamers, polyesteramides, polyurethanes, polyacetals, polyorthocarbonates, polyphosphazenes, polyhydroxyvalerates, polyalkylene succinates, poly (malic acid), polyamino acids, chitin, chitosan, gelatin, collagen, atelocollagen, dextran, proteins, and polyorthoesters, and copolymers, terpolymers and combinations and mixtures thereof.

66. The particle of claim 53, wherein the biocompatible polymer is polymerized from monomers during the particle preparation.

67. The particle of claim 53, wherein the biologically active agent is selected from the group consisting of antineoplastics, blood products, biological response modifiers, anti-fungals, antibiotics, hormones, vitamins, proteins, peptides, enzymes, dyes, anti-allergies, anti-coagulants, circulatory agents, metabolic potentiators, antituberculars, antivirals, antianginals, anti-inflammatories, antiprotozoans, antirheumatics, narcotics, opiates, diagnostic imaging agents, cardiac glycosides, neuromuscular blockers, sedatives, anesthetics, paramagnetic particles, radioactive particles, antibodies, antibody fragments, genetic material, contrast agents, dyes and derivatives and combinations thereof.

68. The particle of claim 67, wherein the biologically active agent is selected from the group consisting of cisplatin, carboplatin, oxaliplatin, doxorubicin, camptothecin, taxol, mitomycin, verapamil, folate anatagonists and methotrexate.

69. The particle of claim 53, wherein the biologically active agent is a prodrug.

70. A kit for administering a biologically active substance to a patient comprising a unit dose of magnetically targetable particles of claim 53, and a vehicle enabling the administration of the particles.

71. A kit for administering a biologically active agent comprising;
a) a first container comprising a unit dose of magnetically targetable particles of claim 53, each particle including a ratio of magnetic component to polymer in the range from about 99:1 to about 30:70; and
b) a second container with a solution comprising one or more excipients.

72. The kit of claim 71, further comprising a biologically compatible polymer.

73. The kit of claim 71, wherein the excipient is selected from the group consisting of mannitol, sorbitol, glucose, sucrose, sodium carboxymethylcellulose, polyethylene glycol, polyvinyl pyrrolidone and combinations thereof.

74. The kit of claim 71, wherein the unit dose of magnetically targetable particles has been further sterilized by a method selected from the group consisting of gamma irradiation, dry heat and electron beam.

75. The kit of claim 71, wherein the solution comprising an excipient has been sterilized by means of autoclave.

76. A method of sterilizing the magnetically targetable particles of claim 53 comprising irradiating the particles with a sterilizing amount of gamma irradiation.

77. A method for the localized in vivo delivery of a biologically active agent comprising:
a) suspending the magnetically targetable particle of claim 53 in a vehicle for injection
b) injecting the vehicle loaded with the magnetically targetable particle; and
c) establishing a magnetic field at a desired site of sufficient strength to guide and retain a portion of the magnetically targetable particles.

78. The method of claim 77, wherein the injecting step is via intra-arterial.

79. The method of claim 77, wherein the desired site includes a tumor.

80. The method of claim 77, wherein the biologically active agent is selected from the group consisting of a diagnostic agent, a therapeutic agent, an agent that functions as a therapeutic and a diagnostic agent, and combinations thereof.
